(19) United States
(12) Patent Application Publication
(10) Pub. No.: US 2025/0223245 A1
(43) Pub. Date: Jul. 10, 2025

(54) PROCESS AND DEVICE FOR PRODUCING LIQUID METHANE

(71) Applicant: L'Air Liquide, Societe Anonyme pour l'Etude et l’Exploitation des Procedes Georges Claude, Paris (FR)

(72) Inventors: Laura AYRAULT, Sassenage (FR); Mathieu ROIG, Sassenage (FR)

(21) Appl. No.: 19/016,247

(22) Filed: Jan. 10, 2025

(30) Foreign Application Priority Data
Jan. 10, 2024 (FR) FR 2400220

(51) Int. Cl. C07C 7/04; B01D 5/00; F25J 3/02

(52) U.S. Cl. CPC C07C 7/04; B01D 5/006; B01D 5/0075; F25J 3/0233; F25J 2200/06; F25J 2215/04; F25J 2215/60; F25J 2270/42

(57) ABSTRACT

Process and device for producing nitrogen-purified liquid methane from a feed gas stream containing at least 98 mol % of methane and between 0.1 and 2 mol % of nitrogen, in which the feed gas stream is cooled to a temperature of between 110 K and 200 K in a first heat exchanger in countercurrent with a stream of nitrogen vapour to produce a cooled stream, a bath of liquid nitrogen is in heat exchange with the cooled stream via a second heat exchanger through which the cooled stream passes, the liquid nitrogen bath producing nitrogen vapour supplied to the first heat exchanger to produce the cooled stream, the cooled stream leaving the second heat exchanger is introduced into a distillation column with a view to its distillation, and a liquid containing at least 99.5 mol % of methane is withdrawn from the bottom of the distillation column as the final product.

### CROSS-REFERENCE TO RELATED APPLICATIONS

This application claims the benefit of priority under 35 U.S.C. § 119 (a) and (b) to French patent application No. FR 2400220, filed Jan. 10, 2024, the entire contents of which are incorporated herein by reference.

### BACKGROUND

The invention concerns a process and device for producing liquid methane.

### Field of the Invention

The invention relates more particularly to a process for producing nitrogen-purified liquid methane from a feed gas stream containing at least 95 mol % of methane and between 0.1 and 5 mol % of nitrogen, in particular at least 98 mol % of methane and between 0.1 and 2 mol % of nitrogen.

### Related Art

Biogas is the gas produced when organic matter breaks down in the absence of oxygen (anaerobic fermentation), also known as methanisation. Biogas can be produced naturally—for example, in marshes or household waste dumps—or it can result from the methanisation of waste in a dedicated reactor under controlled conditions, called a methaniser or digester, and then in a post-digester, similar to the digester, where the methanisation reaction can be taken further.

Biomass refers to any group of organic materials that can be converted into energy through the methanisation process, for example: sewage sludge, manure, agricultural residues, food waste, etc.

Biogas mainly contains methane (CH4) and carbon dioxide (CO2) in proportions that vary according to the method of production and the substrate, but may also contain water, nitrogen, oxygen, hydrogen sulphide (H2S) and volatile organic compounds (VOCs) in lesser proportions.

Depending on the organic matter degraded and the techniques used, the proportions of the components differ, but on average biogas comprises, on a dry gas basis, 30 to 75% methane, 15 to 60% CO2, up to 15% nitrogen, up to 5% oxygen and trace compounds.

More advanced purification of biogas means it can be used more widely. In particular, advanced purification of biogas produces biogas that is purified to natural gas specifications and can be substituted for it; biogas purified in this way is known as “biomethane”. Biomethane thus supplements natural gas resources with a renewable component produced in the heart of the regions; it can be used for exactly the same purposes as natural gas of fossil origin. It can be used to supply a natural gas network or a vehicle filling station, or it can be liquefied for storage and transport in the form of liquid natural gas (bioGNL), etc.

The “classic” purification process (membrane or water scrubbing column) can eliminate CO2 up to ˜1 to 2.5%. An adsorption stage at ambient temperature reduces its concentration to <50 ppm. On the other hand, air gases tend to remain with the methane during the purification stages. The remaining air can be eliminated, for example by means of a catalyst, until a small quantity remains, up to 1 to 2%.

When very high methane purity(>99.9%, for example) and a good recovery rate(>99%) are required, these purification bricks do not make it possible to achieve these objectives. The present invention describes a solution for liquefying biomethane and purifying it to nitrogen using the same Installation. The invention can also be applied to the liquefaction of any source of methane containing nitrogen, for example natural gas or a mixture of natural gas and biomethane.

The document published under number EP 3465035 A1 describes a cryogenic distillation process as a step in the purification of biogas containing relatively high quantities of air (nitrogen and oxygen) (between 3 and 50 mol % nitrogen and oxygen). The targeted methane concentrations at the outlet are those compatible with the specifications for reinjection into the natural gas network, or for vehicle fuel, which corresponds to CH4 mol>97.5%. In particular, the design of the cryogenic distillation solves the problem of explosive atmospheres that could be created with conventional distillation when biogas rich in air gases needs to be purified.

The documents published under numbers FR 2971331 A1 and FR 2971332 A1 also propose cryogenic distillation processes for the purification of biogas containing between 65 and 97% methane, the remainder being air. To prevent the formation of an explosive mixture in the column, the gaseous and/or liquid mixtures in the distillation column are diluted by reinjecting some of the liquid methane into the column bottom or by injecting gaseous nitrogen from an external source into the bottom.

The prior art described above proposes cryogenic distillation solutions as a purification stage for biogas containing relatively high quantities of air (nitrogen and oxygen) in order to avoid the risk of explosion inside a distillation column. However, these documents do not concern the liquefaction of methane as such, in particular with the elimination of residual nitrogen, or the optimisation of a design for this purpose.

### SUMMARY OF THE INVENTION

To this end, the process according to the invention, which otherwise conforms to the generic definition given in the preamble above, is essentially characterised in that:
the feed gas stream is cooled to a temperature of between 110 K and 200 K, in particular between 130 K and 150 K, in a first heat exchanger in counter-current to a stream of nitrogen vapour to produce a cooled stream,
a liquid nitrogen bath is in heat exchange with the cooled stream via a second heat exchanger through which the cooled stream passes, the liquid nitrogen bath producing nitrogen vapour supplied to the first heat exchanger to produce the cooled stream,
the cooled stream leaving the second heat exchanger is introduced into a distillation column for distillation, and
a liquid containing at least 99.5 mol %, preferably at least 99.9 mol %, of methane is withdrawn from the bottom of the distillation column as the final product.

In addition, embodiments of the invention may comprise one or more of the following features:
The feed gas stream contains at least 99 mol % of methane, in particular at least 99.5 mol % of methane, and between 0.1 and 1 mol % of nitrogen, in particular between 0.1 and 0.5 mol % of nitrogen.
Before being introduced into the distillation column, the cooled stream leaving the second heat exchanger passes through a boiler in the column.
A vapour phase depleted in methane and enriched in nitrogen relative to the feed gas stream is withdrawn from the top of the distillation column and sent to a condenser in which the vapour phase is cooled by heat exchange with liquid nitrogen in order to condense part of the vapour phase, forming a gas enriched in nitrogen relative to the vapour phase and a liquid enriched in methane relative to the vapour phase.
The methane-enriched liquid is returned to the distillation column.
The liquid nitrogen is vaporised in the condenser forming nitrogen vapour which is supplied to the first heat exchanger to produce the cooled stream.
The condenser comprises a plate heat exchanger immersed in liquid nitrogen.
The liquid withdrawn from the bottom of the distillation column is cooled in a third heat exchanger by heat exchange with liquid nitrogen, liquid nitrogen being vaporised to form nitrogen vapour which is supplied to the first heat exchanger to produce the cooled stream.
The cooled stream is introduced at the top of the distillation column.

The invention also relates to an device for producing nitrogen-purified liquid methane from a feed gas stream containing at least 95 mol % of methane and between 0.1 and 5 mol % of nitrogen, in particular at least 98 mol % of methane and between 0.1 and 2 mol % of nitrogen, the device comprising:
a first heat exchanger configured to cool the feed gas stream to a temperature of between 110 K and 200 K, in particular between 130 K and 150 K, in counter-current with a stream of nitrogen vapour to produce a cooled stream,
a second heat exchanger immersed in a liquid nitrogen bath configured to vaporise the liquid nitrogen by heat exchange with the cooled stream to produce nitrogen vapour,
a distillation column configured to distil the cooled stream and produce a liquid containing at least 99.5 mol %, preferably at least 99.9 mol %, of methane in the column bottom,
a column boiler configured to receive the cooled stream before it is introduced into the distillation column,
a condenser comprising a plate heat exchanger immersed in liquid nitrogen, the condenser being configured to cool a vapour phase withdrawn at the top of the distillation column by heat exchange with the liquid nitrogen, to form a gas enriched in nitrogen with respect to the vapour phase and a liquid enriched in methane with respect to the vapour phase, and to produce nitrogen vapour,
at least one first conduit configured to send the cooled stream leaving the first heat exchanger to the second heat exchanger,
at least one second conduit configured to send the nitrogen vapour leaving the second heat exchanger to the first heat exchanger,
at least one third conduit configured to send the cooled stream leaving the second heat exchanger to the boiler,
at least one fourth conduit configured to send the cooled stream leaving the boiler to the distillation column,
at least one fifth conduit configured to withdraw the vapour phase from the top of the distillation column and send it to the condenser,
at least one sixth conduit configured to return the methane-enriched liquid formed in the condenser to the distillation column,
at least one seventh conduit configured to send the nitrogen vapour leaving the condenser to the first heat exchanger, and
at least one eighth conduit configured to withdraw the liquid containing at least 99.5 mol %, preferably at least 99.9 mol %, of methane from the bottom of the column as the final product.

Depending on other possible features:
The device comprises a third heat exchanger immersed in a liquid nitrogen bath configured to cool the liquid withdrawn from the bottom of the distillation column and vaporise the liquid nitrogen to produce nitrogen vapour, and at least one ninth conduit configured to send the nitrogen vapour produced to the first heat exchanger.
The appliance includes a liquid nitrogen storage connected to the second heat exchanger and/or the third heat exchanger and/or the condenser.

The present invention provides a solution for liquefying a methane-rich stream, in particular from biogas purification and/or natural gas, and containing nitrogen, in particular a residual nitrogen content, and purifying it for nitrogen efficiently in a single installation. A gas stream containing at least 95 mol %, in particular at least 99.5 mol %, of methane and 5 mol % or less of nitrogen, in particular 0.5 mol % or less of nitrogen, can be liquefied and purified by cryogenic distillation. The creation of an explosive atmosphere due to oxygen is not a concern.

The invention may also relate to any alternative device or process comprising any combination of the above or below features within the scope of the claims.

### BRIEF DESCRIPTION OF THE FIGURES

Other features and advantages will become apparent from the description below, with reference to the figure.

FIG. 1 schematically illustrates an example of an device and a process according to the invention.

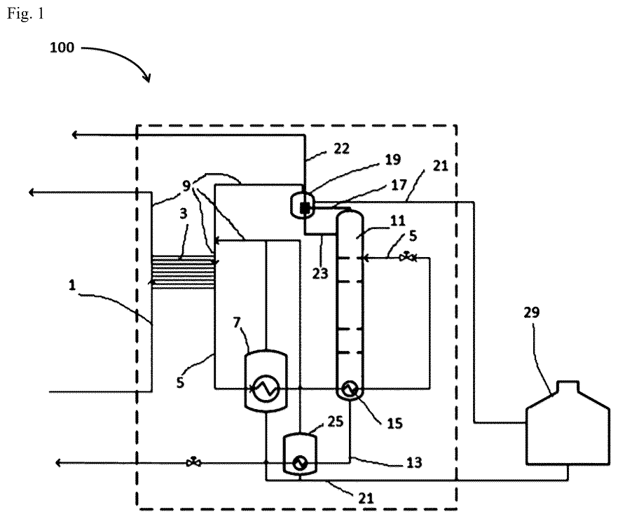

### DETAILED DESCRIPTION OF THE INVENTION

A gas stream 1 is fed into the device 100 to produce liquid methane 13 purified of nitrogen. The feed gas stream 1 contains at least 95 mol % of methane and up to 5 mol % of nitrogen. In particular, the feed gas stream 1 contains at least 98 mol % of methane and between 0.1 and 2 mol % of nitrogen. More particularly, the feed gas stream 1 contains at least 99 mol % of methane and between 0.1 and 1 mol % of nitrogen. Even more particularly, the feed gas stream 1 contains at least 99.5 mol % of methane and between 0.1 and 0.5 mol % of nitrogen.

The feed gas stream 1 is, for example, biomethane produced from biogas purification, for example by membrane separation, by washing column, for example with water or solvent, by cryogenic distillation and/or by adsorption, for example at ambient temperature or at modulated temperature and/or pressure (temperature and/or pressure swing adsorption).

The feed gas stream 1 may be natural gas or a mixture of natural gas and biomethane.

The feed gas stream 1 is cooled in a first heat exchanger 3 in counter-current to a stream of nitrogen vapour 9. The first heat exchanger 3 is, for example, of the Brazed Aluminium Heat Exchangers (BAHX) type.

The cooled stream 5 leaving the first heat exchanger 3 is at a temperature of between 110 K and 200 K, in particular between 130 K and 150 K.

The cooled stream 5 leaving the first heat exchanger 3 passes through a second heat exchanger 7 which is, for example, immersed in a bath of liquid nitrogen, in which the cooled stream 5 and the bath of liquid nitrogen are in heat exchange. The cooled stream 5 heats the liquid nitrogen bath in the second heat exchanger 7 by heat exchange. This step lowers the temperature of the cooled gas stream 5 by approximately 1 K, thus enabling part of the liquid nitrogen to be vaporised and incorporated in vapour form into the first heat exchanger 3, while making the most of the latent heat of vaporisation. The nitrogen vapour 9 produced is returned to the first heat exchanger 3 to cool the feed gas stream 1 by heat exchange.

The use of a nitrogen bath avoids two-phase heat exchangers and direct contact between the liquid nitrogen and the gas stream 5.

The level of the bath is kept constant by adding liquid nitrogen from source 29 to compensate for the loss of level due to vaporisation.

The cooled stream 5 leaving the second heat exchanger 7 is introduced into a distillation column 11 for distillation. The cooled stream 5 can be introduced into a zone of the distillation column 11, depending in particular on its composition and the desired operating conditions, in particular in the upper part of the distillation column 11.

Preferably, before being introduced into the distillation column 11, the cooled stream 5 leaving the second heat exchanger 7 passes through a boiler 15 in the column. The cooled stream 5 recovers cold power to finish cooling, while delivering hot power to the boiler 15, by heat exchange in the boiler 15. The boiler 15 in the column may be located in the bottom of the column 11 or outside the column 11.

As illustrated, a vapour phase 17 depleted in methane and enriched in nitrogen compared with the feed gas stream 1, 5 is drawn off at the top of the distillation column 11. This vapour phase 17 may be sent to a condenser 19, which is separate from the distillation column 11, for example. Condenser 19 may comprise or may consist of a plate heat exchanger bathed in liquid nitrogen 21. The vapour phase 17 is cooled by heat exchange with the liquid nitrogen 21, preferably in a liquid nitrogen bath 21, in the condenser 19 in order to condense part of the vapour phase 17, forming a liquid 23 enriched in methane and depleted in nitrogen relative to the vapour phase 17 and a gas 22 enriched in nitrogen and depleted in methane relative to the vapour phase 17.

The methane-enriched liquid 23 is sent to the top of column 11 to form a reflux liquid. The nitrogen-enriched gas 22 is evacuated to a vent line, where the residual methane and/or the residual cold power can be recovered.

In condenser 19, liquid nitrogen 21 is vaporised 9 and fed back into the first heat exchanger 3 to cool the feed gas stream 1 by heat exchange.

A liquid 13 containing at least 99.5 mol %, preferably at least 99.9 mol % or even at least 99.99 mol % of methane is drawn off in the tank or at the bottom of the distillation column 11 as the final product. The liquid 13 withdrawn can be cooled in a third heat exchanger 25 by heat exchange with liquid nitrogen 21, in particular a liquid nitrogen bath. The third heat exchanger 25 is preferably a plate heat exchanger bathed in a liquid nitrogen bath. In particular, the liquid 13 is in the saturated liquid state at a temperature of about 130 K. Its temperature is lowered to approximately 110 K via the third heat exchanger 25. Cooling in the third heat exchanger 25 prevents the liquefied methane from vaporising. The liquid nitrogen 21 vaporised 9 in this way can be reused in the first heat exchanger 3 to cool the feed gas stream 1 by heat exchange.

The process according to the invention, in particular the cooling of the streams and the distillation, is preferably carried out in a cold box (dotted frame in the figure). The device 100, in particular the heat exchangers 3, 7, 19, 25 and the distillation column 11, is preferably kept in the cold box.

The liquid nitrogen 21 used in the process according to the invention is preferably pressurised in order to avoid a temperature which would cause the methane to freeze.

The process according to the invention makes it possible to pool the refrigerant source for the condenser, (pre-) cooling and liquefaction, and to integrate the stream of refrigerant (nitrogen) vapour in the same (pre-)cooling exchanger (the first heat exchanger 3).

It is possible to achieve a methane molecule recovery rate of over 99.9%, while minimising refrigerant consumption.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing i.e. anything else may be additionally included and remain within the scope of “comprising.” “Comprising” is defined herein as necessarily encompassing the more limited transitional terms “consisting essentially of” and “consisting of”; “comprising” may therefore be replaced by “consisting essentially of” or “consisting of” and remain within the expressly defined scope of “comprising”.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

1. A process for the production of nitrogen-purified liquid methane from a feed gas stream containing at least 95 mol % of methane and between 0.1 and 5 mol % of nitrogen, wherein:
the feed gas stream is cooled to a temperature of between 110 K and 200 K in a first heat exchanger in counter-current to a stream of nitrogen vapour to produce a cooled stream,
a liquid nitrogen bath is in heat exchange with the cooled stream via a second heat exchanger through which the cooled stream passes, the liquid nitrogen bath producing nitrogen vapour supplied to the first heat exchanger to produce the cooled stream,
the cooled stream leaving the second heat exchanger is introduced into a distillation column for distillation, and
a liquid containing at least 99.5 mol % of methane is withdrawn from the bottom of the distillation column as the final product.

2. The process according to claim 1, wherein, before being introduced into the distillation column, the cooled stream leaving the second heat exchanger passes through a boiler of the column.

3. The process according to claim 1, wherein a vapour phase depleted in methane and enriched in nitrogen relative to the feed gas stream is drawn off at the head of the distillation column and sent to a condenser in which the vapour phase is cooled by heat exchange with liquid nitrogen in order to condense part of the vapour phase forming a gas enriched in nitrogen relative to the vapour phase and a liquid enriched in methane relative to the vapour phase.

4. The process according to claim 3, wherein the methane-enriched liquid is returned to the distillation column.

5. The process according to claim 3, wherein the liquid nitrogen is vaporised in the condenser forming nitrogen vapour which is supplied to the first heat exchanger to produce the cooled stream.

6. The process according to claim 3, wherein the condenser comprises a plate heat exchanger immersed in liquid nitrogen.

7. The process according to claim 1, wherein the liquid withdrawn from the bottom of the distillation column is cooled in a third heat exchanger by heat exchange with liquid nitrogen, liquid nitrogen being vaporised to form nitrogen vapour which is supplied to the first heat exchanger to produce the cooled stream.

8. The process according to claim 1, wherein the cooled stream is introduced into the upper part of the distillation column.

9. A device for producing nitrogen-purified liquid methane from a feed gas stream containing at least 95 mol % of methane and between 0.1 and 5 mol % of nitrogen, the device comprising:
a first heat exchanger configured to cool the feed gas stream to a temperature of between 110 K and 200 K in counter-current to a nitrogen vapour stream to produce a cooled stream,
a second heat exchanger immersed in a liquid nitrogen bath configured to vaporise the liquid nitrogen by heat exchange with the cooled stream to produce nitrogen vapour,
a distillation column configured to distil the cooled stream and produce a liquid containing at least 99.5 mol % of methane from the bottom of the column,
a column boiler configured to receive the cooled stream before it is introduced into the distillation column,
a condenser comprising a plate heat exchanger immersed in liquid nitrogen, the condenser being configured to cool a vapour phase withdrawn at the top of the distillation column by heat exchange with the liquid nitrogen, to form a gas enriched in nitrogen with respect to the vapour phase and a liquid enriched in methane with respect to the vapour phase, and to produce nitrogen vapour,
at least one first conduit configured to send the cooled stream leaving the first heat exchanger to the second heat exchanger,
at least one second conduit configured to send the nitrogen vapour leaving the second heat exchanger to the first heat exchanger,
at least one third conduit configured to send the cooled stream leaving the second heat exchanger to the boiler,
at least one fourth conduit configured to send the cooled stream leaving the boiler to the distillation column,
at least one fifth conduit configured to withdraw the vapour phase from the top of the distillation column and to send the withdrawn vapor phase to the condenser,
at least one sixth conduit configured to return the methane-enriched liquid formed in the condenser to the distillation column,
at least one seventh conduit configured to send the nitrogen vapour leaving the condenser to the first heat exchanger, and
at least one eighth conduit configured to withdraw the liquid containing at least 99.5 mol % of methane from the bottom of the column as the final product.

10. The device according to claim 9 comprising a third heat exchanger immersed in a liquid nitrogen bath configured to cool the liquid withdrawn from the bottom of the distillation column and vaporise the liquid nitrogen to produce nitrogen vapour, and at least one ninth conduit configured to send the nitrogen vapour produced to the first heat exchanger.

11. The device according to claim 9 comprising a liquid nitrogen storage connected to the second heat exchanger and/or the third heat exchanger and/or the condenser.
